(19) United States
(12) Patent Application Publication
(10) Pub. No.: US 2017/0283020 A1
(43) Pub. Date: Oct. 5, 2017

(54) REBREATHER CONTROL PARAMETER SYSTEM AND DIVE RESOURCE MANAGEMENT SYSTEM

(71) Applicants: Kevin Gurr, Poole (GB); Nicholas J.K. Bushell, Poole (GB)

(72) Inventors: Kevin Gurr, Poole (GB); Nicholas J.K. Bushell, Poole (GB)

(21) Appl. No.: 15/618,318

(22) Filed: Jun. 9, 2017

Related U.S. Application Data
(63) Continuation-in-part of application No. 15/393,965, filed on Dec. 29, 2016.
Application No. 15/393,965 is a division of application No. 12/862,758, filed on Aug. 25, 2010, now Pat. No. 9,567,047.
(60) Provisional application No. 61/236,426, filed on Aug. 24, 2009.

(51) Int. Cl. B63C 11/24; A62B 9/02; A61M 16/10

(52) U.S. Cl. CPC B63C 11/24; A61M 16/1005; A62B 9/022; B63C 2011/021

(57) ABSTRACT

A method and apparatus for automatically controlling partial pressure of oxygen in the breathing loop of a rebreather diving system. A diver may adjustably select a control parameter to maintain partial pressure of oxygen at a setpoint that varies with ambient pressure and is within a range between a maximum safe partial pressure of oxygen at depth and a minimum safe partial pressure of oxygen for the purpose of biasing the performance of the rebreather either towards minimizing gas venting from the rebreather breathing loop or minimizing decompression time. A method and apparatus for managing and monitoring the use of dive resources in comparison with a target dive time specified by the diver, calculating and indicating remaining dive time based on dive resource values and calculating and indicating dive resource values required to meet preselected dive resource end values and dive requirements.

### CROSS-REFERENCE TO RELATED APPLICATIONS

The present application is a continuation-in-part (CIP) of U.S. patent application Ser. No. 15/393,965 filed on Dec. 29, 2016, which is a divisional of U.S. patent application Ser. No. 12/862,758, filed on Oct. 25, 2010, now U.S. Pat. No. 9,567,047 issued on Feb. 14, 2017, which claims the benefit of U.S. Provisional Application No. 61/236,426, filed on Aug. 24, 2009, which is incorporated herein by reference.

### BACKGROUND

### Field of the Invention

The present invention relates generally to diving systems and more particularly to systems and apparatuses for controlling and monitoring rebreather systems and managing the use of dive resources.

### Background Discussion

Self-contained breathing apparatuses used for underwater diving traditionally are categorized either as open circuit systems or rebreather systems. Open circuit systems are relatively simple and well understood in the art but are inefficient and typically require a large breathing gas supply to provide a reasonable dive time. Each breath inhaled by a diver from an open circuit system is exhaled to the surrounding environment, wasting any oxygen in the breathing gas that is not metabolized by the diver during the respiration cycle.

Rebreather systems recycle each breath exhaled by a diver by removing the carbon dioxide generated by the diver and replacing the oxygen consumed by the diver during the respiration cycle. Essential components of rebreather systems are a breathing loop comprising a diver's lungs, a mouthpiece, breathing gas supply, a solenoid or flow valve adapted to add breathing gas to the breathing loop, a pressure regulator for the breathing gas supply, a scrubber canister adapted to remove exhaled carbon dioxide, a counterlung, a valve adapted to vent or purge gas from the breathing loop if the total gas pressure in the breathing loop exceeds a preselected pressure value, flexible, gas-impermeable hoses connecting the various components and uni-directional check valves to control the flow of breathing gas through the loop. Rebreather systems may father be categorized as semi-closed circuit systems or closed circuit systems, with each type of rebreather system comprising additional suitable components. Examples of semi-closed circuit and closed circuit rebreather breathing loops are shown in, for example, U.S. Pat. Nos. 5,503,145 and 6,302,106.

In a semi-closed circuit system known in the art, the breathing gas supply commonly comprises one high pressure cylinder containing an oxygen-enriched gas mixture, which typically is introduced to the breathing loop at a preselected, constant flowrate to replace oxygen consumed during a dive.

In a closed circuit system known in the art, the breathing gas supply commonly comprises one high pressure cylinder containing pure oxygen. A closed circuit system known in the art would additionally typically comprise one or more oxygen sensors adapted to measure the partial pressure of oxygen in the breathing loop during a dive and a computer processor adapted to control the solenoid for the purpose of adding oxygen to the breathing loop as needed to maintain the oxygen partial pressure above a minimum, viable value. A closed circuit system also commonly comprises a high pressure cylinder containing oxygen and an inert gas or mixture of inert gases, also referred to as diluent gases that may be added to the breathing loop via a solenoid and pressure regulator to prevent the counterlung from collapsing due to increasing ambient pressure.

A primary consideration in the design and use of semi-closed and closed circuit rebreather systems is the minimization of the risks of hypoxia, in which the diver is deprived of a life-sustaining oxygen supply, and hyperoxia, which occurs when the diver breathes unsafe elevated oxygen levels. Hypoxia can render a diver unconscious and cause drowning. Hyperoxia may lead to oxygen toxicity, which can have severe physiological effects that can lead to the death of the diver. Oxygen toxicity can manifest as either central nervous system (CNS) oxygen toxicity or pulmonary oxygen toxicity.

Hypoxia and hyperoxia are understood as depending on the partial pressure of oxygen in the breathing gas loop. The partial pressure of oxygen is equal to the product of the total pressure of the gas mixture and the concentration, or fraction, of oxygen in the gas mixture, also expressed as PPO₂=Ptotal*FO₂. Total gas pressure in the breathing loop increases with ambient pressure, which increases by one atmosphere, or bar, per each ten meters of depth. Accordingly, at a constant oxygen concentration, the partial pressure of gas in the breathing loop increases as depth increases and decreases as depth decreases.

Hypoxia occurs when the partial pressure of oxygen in the breathing loop is less than 0.21 bar, which is the ambient partial pressure of oxygen in the atmosphere at sea level. The minimum life-sustaining value of partial pressure of oxygen is 0.16 bar. Maintaining the partial pressure of oxygen in the breathing loop above 0.21 bar will minimize decompression time for the diver. However, pulmonary oxygen toxicity can result from prolonged exposure to oxygen partial pressures above approximately 0.5 bar, and CNS oxygen toxicity becomes a significant risk when the partial pressure of oxygen in the breathing loop is greater than 1.6 bar.

In semi-closed circuit systems, the oxygen-enriched gas mixture is usually added to the breathing loop at a constant rate selected to maintain the partial pressure of oxygen in the breathing loop between 0.21 bar and 1.6 bar based on the estimated oxygen consumption profile of the diver during a dive. However, the rate of addition typically is not automatically adjusted during a dive in response to the actual partial pressure of oxygen in the breathing loop, even though a diver's rate of oxygen consumption, and therefore the oxygen concentration in the breathing loop, may deviate considerably from the rate of consumption estimated prior to the dive. Inequality between the rate of addition and rate of consumption of oxygen can result in depletion or accumulation of oxygen in the breathing loop. Therefore, transient states may occur and persist in semi-closed systems during which the breathing loop contains either hypoxic (i.e. less than 0.21 bar) or hyperoxic (i.e. greater than 1.6 bar) oxygen levels.

Closed circuit rebreather systems provide monitoring and adjustable control of the partial pressure of oxygen in the rebreather breathing loop during a dive. In recognition of the safety and performance concerns outlined above, closed circuit rebreather systems will usually be configured to maintain the partial pressure of oxygen in the breathing loop at a preselected value between 0.21 bar and 1.6 bar for the purpose of reducing the risk of hypoxia and hyperoxia. However, the sensors and automated control systems in closed circuit systems are susceptible to malfunctions, which can lead to hypoxic or hyperoxic oxygen levels in the breathing loop. Additionally, the use of pure oxygen in closed circuit systems creates handling and cleanliness issues with regard to the breathing gas supply.

A further disadvantage of semi-closed circuit and closed circuit systems is that excessive venting of gas from the breathing loop may occur. Semi-closed circuit systems are inherently inefficient because the fixed rate of addition of the oxygen-enriched gas mixture to the breathing loop may exceed the diver's rate of oxygen consumption and cause continual venting or purging of gas from the breathing loop to maintain total gas pressure in the breathing loop below a threshold value.

In closed circuit systems, excessive venting may occur during the ascent phase, during which ambient pressure, and correspondingly total gas pressure and partial pressure of oxygen in the breathing loop, decrease. For example, if the preselected control value for oxygen partial pressure in the breathing loop is set close to 1.6 bar for the purpose of minimizing the diver's decompression time, the closed circuit rebreather control system will increase the addition of oxygen to the breathing loop to compensate for the decrease in partial pressure of oxygen that results from the decrease in ambient pressure. However, the increased volume of gas added to the breathing loop will be vented to maintain total gas pressure in the breathing loop below a threshold value.

The rate of venting of unutilized breathing gas from semi-closed circuit and closed circuit systems may be less than typically experienced with an open circuit system but may nonetheless be sufficient to reduce total dive time or necessitate the use of a larger gas supply to accommodate decompression time during ascent. Additionally, the venting of gas from the breathing loop creates bubbles that may, for example, startle marine life that the diver is attempting to observe, visibly indicate the presence of the diver to observers on the surface or have other undesirable effects.

In view of the limitations of rebreather systems known in the art, a need exists for a rebreather system that provides adjustable control over the partial pressure of oxygen in the breathing loop as well as control over the volume of gas in the breathing loop, and therefore the rate of gas venting from the breathing loop. Such a system would also desirably be free of the disadvantages of using pure oxygen as a breathing gas supply.

Dive monitoring systems known in the art continuously measure, calculate and display parameters such as remaining gas supply, decompression time, partial pressure of oxygen in the breathing loop and depth during a dive to keep a diver informed of the dive profile. These devices are typically configured to display the current status or value of one or more parameters corresponding to dive resources and may also be adapted to assist the diver in pre-planning the dive based on end-of-dive requirements. There is a need for a dive monitoring system configured to continuously inform the diver of remaining dive time based on current dive resources and their usage, inform the diver of the dive resources required based on the dive plan specified by the diver and automatically adjust partial pressure of oxygen in the breathing loop as needed to make it possible for a diver to achieve a target dive time.

### SUMMARY OF INVENTION

An object of this invention is a method for adaptively controlling partial pressure of oxygen in the breathing loop of a rebreather such that the setpoint for partial pressure of oxygen may vary as a function of ambient pressure within a range established by safety considerations and such that a diver can adjust the performance of the rebreather between minimizing gas venting from the rebreather breathing loop and minimizing decompression time. According to the inventive method, the diver specifies values for a control parameter, also known as a Dive Control Parameter (DCP) or Main Control Parameter (MCP), a minimum value of partial pressure of oxygen, a reduction coefficient and concentration of oxygen in a gas supply, and a maximum operating value of partial pressure of oxygen is calculated as a function of ambient pressure, concentration of oxygen in the gas supply and the first reduction coefficient. A setpoint for partial pressure of oxygen is calculated as a function of the Dive Control Parameter, the minimum value of partial pressure of oxygen and the maximum operating value of partial pressure of oxygen. A portion of the gas supply is added to the breathing loop if the partial pressure of oxygen is less than the setpoint for partial pressure of oxygen, or is less than the minimum value of partial pressure of oxygen. In accordance with the invention, minimum and maximum viable partial pressures of oxygen are chosen as limiting values. The gas supply may consist of either pure oxygen having an oxygen concentration of 100%, in which case the value of the setpoint for partial pressure of oxygen is limited to an absolute maximum value selected to avoid increased risk of central nervous system oxygen toxicity or an oxygen-enriched gas mixture having an oxygen concentration of less than 100%, in which case a gas supply is selected to having a concentration of oxygen for which partial pressure of oxygen will not exceed a selected value at a maximum planned dive depth. In some embodiments, the Dive Control Parameter has a value of 0% to 100%, and the reduction coefficient has a value of 0.75 to 0.95.

Another object of this system is an automatic control system configured to adaptively control partial pressure of oxygen in the breathing loop in accordance with the present invention. The automatic control system comprises a rebreather breathing loop and a processor adapted to receive data for partial pressure of oxygen in the rebreather breathing loop, ambient pressure, a selected minimum value of partial pressure of oxygen, a selected concentration of oxygen in the gas supply, a selected value of a first reduction coefficient and a selected value of a first control parameter; calculate a maximum operating value of partial pressure of oxygen as a function of ambient pressure, the selected concentration of oxygen in the gas supply and the selected value of the first reduction coefficient; calculate a setpoint for partial pressure of oxygen as a function of the selected value of the first control parameter, the selected value of a first control parameter and the selected minimum value of partial pressure of oxygen; compare data for partial pressure of oxygen in the rebreather breathing loop with the setpoint for partial pressure of oxygen; and send a signal to add a portion of the gas supply to the rebreather breathing loop if the partial pressure of oxygen in the rebreather breathing loop is less than the setpoint for partial pressure of oxygen.

Another object of this invention is a method for managing the use of dive resources. A target dive time is selected, and a target remaining dive time is calculated from a measurement of elapsed dive time. Dive resource net effects of gas supply duration, scrubber canister duration and battery duration and remaining dive times corresponding to each dive resource net effect are calculated from dive resources corresponding to pressure of a gas supply, scrubber capacity and battery capacity and dive resource usage rates corresponding to decrease in pressure of the gas supply over time, number of injections from the gas supply into the rebreather breathing loop over time and electrical current. Required dive times are calculated from dive variables corresponding to depth, elapsed dive time, partial pressure of oxygen in the rebreather breathing loop, concentration of carbon dioxide in the rebreather breathing loop and temperature of the scrubber canister and dive time limitations corresponding to no decompression limit, decompression time and central nervous system oxygen toxicity percentage. A termination time is identified corresponding to the lowest value of the target remaining dive time and each of the remaining dive times. In accordance with the invention warning indicator is displayed if the required dive time is equal to or greater than the termination time, and otherwise the termination time is displayed. In some embodiments, if the required dive time is equal to or greater than the termination time, the setpoint for partial pressure of oxygen is reduced within safety limits to increase termination time and enable completion of the dive as planned.

Another object of this system is a dive resource management system configured to monitor and manage dive resources, dive resource usage rates, dive resource net effects, dive variables and dive time limitations. In certain embodiments, the dive resource management system displays information and warnings corresponding to the status of dive resources and dive time limitations relative to a target dive time. In certain embodiments, the dive resource management system, automatically adjusts the setpoint for partial pressure of oxygen to conserve dive resources if required dive time limitations exceeds target remaining dive time.

The object of the present invention may further be described in the following paragraphs elaborating methods, systems and apparatuses of the present disclosure.

A method for automatically controlling partial pressure of oxygen in a rebreather breathing loop may comprise storing a minimum value of partial pressure of oxygen, measuring partial pressure of oxygen in the rebreather breathing loop, adding a portion of a gas supply to the rebreather breathing loop if partial pressure of oxygen in the rebreather breathing loop is less than the minimum value of partial pressure of oxygen, storing a value of a first reduction coefficient; measuring ambient pressure; calculating a maximum operating value of partial pressure of oxygen as the product of ambient pressure, concentration of oxygen in the gas supply and the first reduction coefficient; storing a value of a first control parameter; calculating a setpoint for partial pressure of oxygen as the sum of the minimum value of partial pressure of oxygen and the product of the first control parameter and the absolute value of the difference between the maximum operating value and minimum value of partial pressure of oxygen; and adding gas supply to the rebreather breathing loop if partial pressure of oxygen in the rebreather breathing loop is less than the setpoint for partial pressure of oxygen.

The value of the first reduction coefficient may be from 0.75 to 0.95.

The value of the first control parameter may be from 0% to 100%.

The gas supply is oxygen, may further comprise storing an absolute maximum value of partial pressure of oxygen, and limiting the setpoint for partial pressure of oxygen to the absolute maximum value of partial pressure of oxygen.

The gas supply may be an oxygen-enriched gas mixture, further comprising selecting a gas supply having a concentration of oxygen for which partial pressure of oxygen in the gas supply will not exceed a selected value at a maximum planned dive depth.

A method for managing the use of dive resources may comprise storing a target dive time, measuring an elapsed dive time, calculating a target remaining dive time, measuring a dive resource, measuring a dive resource usage rate, calculating a dive resource net effect, measuring a dive variable; calculating a dive time limitation, comparing the target remaining dive time and each dive resource net effect, and identifying a termination time as the lowest value of the target remaining dive time and each dive resource net effect.

The method may further comprise displaying the lowest dive resource net effect if the target remaining dive time is not the termination time and the dive time limitation is less than the termination time.

The method may further comprise displaying the absolute value of the difference between the termination time and the lowest dive resource net effect if the target remaining dive time is the termination time and the dive time limitation is less than the termination time.

The method may further comprise displaying a first warning indicator if the dive time limitation is equal to or greater than the termination time.

The method may further comprise adjusting a setpoint for partial pressure of oxygen in a rebreather breathing loop if the dive time limitation is greater than the termination time so that the termination time will be equal to the target remaining dive time.

The dive resource may constitute the pressure of a gas supply, the scrubber capacity or the battery capacity. The dive resource usage rate may decrease in pressure of the gas supply over time, the number of injections from the gas supply into the rebreather breathing loop over time or electrical current. The dive resource net effect may be the gas supply duration, the scrubber canister duration or the battery duration. The dive variable may constitute depth, elapsed dive time or partial pressure of oxygen in the rebreather breathing loo, and the dive time limitation may be no decompression limit, decompression time or central nervous system oxygen toxicity percentage.

The dive resource may further comprise calculating the gas supply duration as a function of the pressure of the gas supply and the decrease in pressure of the gas supply over time or the number of injections from the gas supply into the rebreather breathing loop over time.

The method may further comprise defining a reserve gas supply, displaying a second warning indicator if the termination time corresponds to the gas supply duration where the target remaining dive time is greater than the gas supply duration, the target remaining dive time is less than the sum of the gas supply duration and the reserve gas supply duration, and/or the dive time limitation is less than the sum of the gas supply duration and the reserve gas supply duration. The method may further comprise displaying a third warning indicator if the termination time corresponds to the gas supply duration, the target remaining dive time is greater than the sum of the gas supply duration and the reserve gas supply duration, and/or the dive time limitation is less than the sum of the gas supply duration and the reserve gas supply duration.

The dive resource may be scrubber capacity. The dive resource usage rate may be the number of injections from the gas supply into the rebreather breathing loop over time, and the dive resource net effect may be the scrubber canister duration and the dive variable may be concentration of carbon dioxide in the rebreather breathing loop or temperature of the scrubber canister. The method may further comprise calculating the scrubber canister duration as a function of either the number of injections from the gas supply into the rebreather breathing loop over time, the concentration of carbon dioxide in the rebreather breathing loop or the temperature of the scrubber canister.

The dive resource may be battery capacity; the dive resource usage rate may be electrical current and the dive resource net effect may be battery duration.

The method may further comprise: calculating the battery duration as a function of the battery capacity and the electrical current.

The dive variables may be depth and elapsed dive time and the dive time limitation may be no decompression limit or decompression time.

The method may further comprise calculating no decompression limit or decompression time as a function of depth and elapsed dive time.

The dive variables may be elapsed dive time and partial pressure of oxygen in the rebreather breathing loop and the dive time limitation may be central nervous system oxygen toxicity percentage.

The method may further comprise: calculating central nervous system oxygen toxicity percentage as a function of elapsed dive time and partial pressure of oxygen in the rebreather breathing loop.

An automatic control system for a rebreather breathing loop may comprise a gas supply; a gas supply pressure regulator, a scrubber canister, a counterlung, a first sensor adapted to measure ambient pressure, a second sensor adapted to measure partial pressure of oxygen, a first valve adapted to add the gas supply to the rebreather breathing loop, a power source, connecting hoses, check valves adapted to control the direction of flow of gas in the rebreather breathing loop, and a processor adapted to receive data for partial pressure of oxygen in the rebreather breathing loop, ambient pressure, a selected minimum value of partial pressure of oxygen, a selected concentration of oxygen in the gas supply, a selected value of a first reduction coefficient and a selected value of a first control parameter, calculate a maximum operating value of partial pressure of oxygen as a function of ambient pressure, the selected concentration of oxygen in the gas supply and the selected value of the first reduction coefficient, calculate a setpoint for partial pressure of oxygen as a function of the selected value of the first control parameter, the selected value of a first control parameter and the selected minimum value of partial pressure of oxygen, compare data for partial pressure of oxygen in the rebreather breathing loop with the setpoint for partial pressure of oxygen, and send a signal to add a portion of the gas supply to the rebreather breathing loop if the partial pressure of oxygen in the rebreather breathing loop is less than the setpoint for partial pressure of oxygen.

A dive resource management system for a rebreather breathing loop may comprise a gas supply, a gas supply pressure regulator, a scrubber canister, a counterlung, a first sensor adapted to measure ambient pressure, a pressure transducer adapted to indicate depth as a function of ambient pressure, a second sensor adapted to measure partial pressure of oxygen, a first valve adapted to add the gas supply to the rebreather breathing loop, a power source, a clock, connecting hoses, check valves adapted to control the direction of flow of gas in the rebreather breathing loop, and a processor adapted to receive data for target dive time, elapsed dive time, pressure of a gas supply, battery capacity, decrease in pressure of the gas supply over time, electrical current, depth and or partial pressure of oxygen in the rebreather breathing loop calculate target remaining dive time, gas supply duration, battery duration, no decompression limit, decompression time, and central nervous system oxygen toxicity percentage and remaining dive time corresponding to gas supply duration, battery duration, no decompression limit, decompression time and central nervous system oxygen toxicity percentage, and identify a dive termination time.

The processor may be further adapted to send a signal to display the dive termination time or a signal to display a warning indicator if the dive termination time is not equal to the target remaining dive time, further comprising: a graphical display.

The processor may be further adapted to receive data for scrubber capacity, concentration of carbon dioxide in the rebreather breathing loop, number of injections from the gas supply into the rebreather breathing loop over time and temperature of the scrubber canister and calculate scrubber canister duration and remaining dive time corresponding to scrubber canister duration, further comprising: a third sensor adapted to measure concentration of carbon dioxide; a fourth sensor adapted to measure temperature of the scrubber canister.

The processor may be further adapted to send a signal to adjust a setpoint for partial pressure of oxygen in the rebreather breathing loop if the required dive time is greater than the termination time so that the termination time will be equal to the target remaining dive time.

An apparatus comprising a loop control valve that includes (i) a loop port configured for coupling to receive a gas feed via a one-way valve from an exhale counterlung of a rebreather loop, and (ii) one or more vent ports configured to selectively vent a volume of gas from the rebreather loop; and a subsystem configured to vary a loop pressure at which the loop control valve is operable to selectively vent the volume of gas from the rebreather loop.

The loop control valve may comprise a valve stop that selectively gates the flow of the gas from the rebreather loop to the vent port, and wherein the subsystem comprises at least one mechanical component configured to vary a force applied to the valve stop according to the orientation of the loop control valve.

The at least one mechanical component may be configured to vary the force applied to the valve stop based on variation in the component of the weight of at least a portion of the at least one mechanical component that acts on the valve stop.

The subsystem may comprise a processor that is configured to detect an accelerometer signal and to selectively gate the loop control valve to vent the volume of gas from the rebreather loop based on at least the accelerometer signal.

Other systems, methods, features, and advantages of this invention will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF DRAWINGS

These and other features, aspects and advantages of this invention will become better understood with regard to the following description, appended claims and accompanying drawings where:

FIG. 1 shows a flowchart of a method embodying features of the present invention for controlling partial pressure of oxygen in the breathing loop of a rebreather system;

FIG. 2 shows an exemplary graphical representation of dive depth plotted as a function of time;

FIG. 3 shows an exemplary graphical representation of setpoint for partial pressure of oxygen plotted as a function of time;

FIG. 4 shows an exemplary graphical representation of measured partial pressure of oxygen plotted as a function of time;

FIG. 5 shows an exemplary graphical representation of control error difference between setpoint for partial pressure of oxygen and measured partial pressure of oxygen plotted as a function of time;

FIG. 6 shows an exemplary graphical representation of the operating range of partial pressure of oxygen as a function of ambient pressure, control parameter value and setpoint for partial pressure of oxygen;

FIG. 7 shows a flowchart of a method embodying features of the present invention for managing dive resources, dive resource usage rates, dive resource net effects, dive variables and dive time limitations;

FIG. 8 shows an exemplary graphical representation of a warning indication corresponding to the viability of a dive that is displayed to a diver;

FIG. 9 shows an exemplary graphical representation of a warning indication corresponding to the viability of a dive that is displayed to a diver;

FIG. 10 shows an exemplary graphical representation of a warning indication corresponding to the viability of a dive that is displayed to a diver;

FIG. 11 shows an exemplary graphical representation of dive resource net effects and dive time limitations that are displayed to a diver;

FIG. 12 shows an exemplary graphical representation of dive resource net effects that are displayed to a diver;

FIG. 13 shows an exemplary graphical representation of dive resource net effects that are displayed to a diver;

FIGS. 14A-C schematically depict an illustrative loop control valve (LCV) that provides for venting loop overpressure while compensating for the hydrostatic effect, in accordance with some embodiments.; and

FIGS. 15A and 15B are operational diagrams of the functionality of the Loop Control Valve in two (2) different orientations.

### DETAILED DESCRIPTION OF EMBODIMENTS

An electro-mechanical rebreather system in accordance with certain embodiments includes various mechanical features and a resource management system. The mechanical features comprise a breathing loop including a front case, a back case, a harness, a canister that houses an Absorbent cartridge an exhale counterlung, an inhale counterlung, a Buoyancy Control Device (BCD), a combined Dive-Surface Valve (DSV) and Bail-out Valve (BOV), a power source, connecting/breathing hoses, a gas supply pressure regulator and a gas supply consisting of either a single high-pressure source containing an oxygen-enriched gas mixture or a source containing pure oxygen in conjunction with a source containing diluent gas, a Loop Control Valve (LCV) and LCV counterlung port/tube, oxygen and CO2 sensors. The electro-mechanical rebreather system comprises three modules, a handset, a Life Support System (LSS) module, and a Sensor Module. The rebreather system further includes an Intelligent Heads Up Display (HUD), and a wireless HP sensor.

In certain embodiments, the rebreather system comprises a passive mechanical addition valve that is adapted to add the gas supply to the rebreather breathing loop at a fixed rate if the ambient pressure exceeds the total gas pressure in the breathing loop by a preselected value. In certain embodiments, the rebreather system comprises a passive mechanical overpressure valve that is adapted to vent gas from the rebreather breathing loop at a fixed rate if the total gas pressure in the breathing loop exceeds ambient pressure by a preselected value. In some embodiments, the preselected value is 25 mbar.

A processor in accordance with certain embodiments of this invention is capable of being programmed by a user with various user-defined parameters and incorporates a display and buttons, keys, switches or similar articles suitable for inputting information to the processor. The processor is implemented, in accordance with the invention, as a microprocessor, microcontroller, digital signal processing circuit or firmware programmed to receive measurement data and perform calculations. The processor includes sensor ports for receiving signals from pressure transducers, oxygen sensors and carbon dioxide sensors, for example. The processor also includes output ports for transmitting signals to components of the rebreather system, including solenoid valves. Suitable processors can be implemented from conventional, commercially-available components having an input and an output bus and including an arithmetic computational ability. Various suitable processors are manufactured by, for example, Renesas and Microchip.

As mentioned previously, the electro-mechanical rebreather system comprises three modules, the Handset, the LSS Module and the Sensor Module. The following description is divided by module, detailing algorithms implemented on those modules. Model classes are given in Unified Modelling Language (UML) notation, with pseudo-code or ‘C’ code given for class operations.

### Handset

The Handset is configured for placement on the wrist and/or forearm of the diver. Among the functions the Handset performs are tissue modeling/decompression, resource management, breathing detection, auto wake at depth, HP usage and depth rate averaging.

The Handset module uses a body/tissue model class (CBody) comprising multiple tissue classes (CTissueModel) and a Central Nervous System (CNS) model plus an interface to the decompression profile and No-Stop-Time (NST, also referred to as No Decompression Limit (NDL)) for a diver object (CDiver instance).

Among the operations of the CBody class can undertake are, to update all tissues (of the body instance) in the given environment (time) and inspired gas mix

TABLE 1
CBody::UpdateTissues(env: CEnvironment, gas: CGasMix)

LOOP tissue (of body.tissues)

tissue.ExposeToInspiredGas(gas, env.GetAmbientPressure( ),
env.GetTime( ) − time)

END LOOP
time = env.GetTime( )

and return the current maximum tolerated pressure of the body instance.

TABLE 2
CBody::GetToleratedPressure( ) : Float

MaxPAmbTol = 0
LOOP tissue (of body.tissues)

MaxPAmbTol = MAX(MaxPAmbTol,
tissue.GetToleratedAmbientPressure( ))

END LOOP
return MaxPAmbTol

Among the operations of the Tissue Model Class performs are to expose the tissue instance to an inspired gas mix at the given ambient pressure for the given time

TABLE 3
CTissueModel::ExposeToInspiredGas(CGasMis *InspiredGas, float fAmbientPressure, float fTime)

fPartialPressure += (InspiredGas.fFractionOfN2*(fAmbientPressure-WATER_VAPOUR_PRESSURE)−

fPartialPressure)*(1.0−pow(2.0, −(fTime/fHalfTime)))

where fPartialPressure is the current partial pressure N₂ in the tissue and the half-time fHalfTime is fixed for each tissue.

The Tissue Model Class also returns the maximum tolerated pressure of the tissue instance.

TABLE 4
CTissueModel::GetToleratedAmbientPressure( ) : Float

P Amb Tol = (Pig − a).b, where Pig is the Partial Pressure of Inert Gas
(in the tissue at a given time) and a, b are components of a
CPAmbTolGradiant object as returned from the
CBuhlmannTissueThresholdModel object

### NST Model

The NST is updated in the given environment, with the given breathing equipment using a bisection algorithm.

TABLE 5
float fTimeMin = 0.0;
float fTimeMax = NST_MAX;
float fTimeMid = NST_MAX/2;
unsigned short int usiIter = 0;
// Get the equipment's inspired gas mix (either live or calculated).
// Note: this assumes EQUIP_CALCULATE_INSPIRED_GAS( ) is independent of the bottom time in
CDiveStatus

| | |
|---|---|
| // | (as updated by bodyPAmbTolEval( )) |

CGasMix gasmix = bLive?EQUIP_GET_INSPIRED_GAS(pequip) : EQUIP_CALCULATE_INSPIRED_GAS(pequip,

envGetAmbientPressure(penv), THIS-

>pr_pds);
// Get starting function evaluations for the bisection
float fMin = bodyPAmbTolEval(THIS, penv, &gasmix, fTimeMin);
float fMax = bodyPAmbTolEval(THIS, penv, &gasmix, fTimeMax);
if (fMin > 0) {

// At time zero (now), a stop is already needed
*pfNST = 0.0;
return;

}
if ((fMin < 0 && fMax < 0) || (fMin > 0 && fMax > 0)) {

// No solution in [0, NST_MAX] - set NST to a ‘huge’ value for trapping by the GUI
*pfNST = NST_HUGE;
return;

}
// Bisect, using the interval [fTimeMin, fTimeMax]
while ((++usiIter < MAX_ITER) && (fTimeMax − fTimeMin > 1)) {

float fMid;
fTimeMid = (fTimeMax + fTimeMin) / 2;
fMid = bodyPAmbTolEval(THIS, penv, &gasmix, fTimeMid);
if ((fMin < 0 && fMid < 0) || (fMin > 0 && fMid > 0)) {

| | |
|---|---|
| fTimeMin | = fTimeMid; |
| fMin | = fMid; |

} else {

| | |
|---|---|
| fTimeMax | = fTimeMid; |

}

}
if (usiIter < MAX_ITER) {

// Round up to the next minute (i.e., will never converge and result in zero)
*pfNST = ceil(fTimeMid);

} else {

// TODO (Tom#4) 2012/03/27 - bodyUpdateNST - when the bisection doesn't converge, NST is zero

(safer)

*pfNST = 0.0;

}

where MAX_ITER is 12, and utilizes the following functions (Tables 6A and 6B):

TABLE 6A
float bodyGetToleratedPressure(CBody* THIS) {

float fMaxPAmbTol = 0.0;
int i;
for (i = 0; i < NUMBER_OF_COMPARTMENTS; i++) {

float PAmbTol = tmGetToleratedAmbientPressure(&THIS−>pr_tmaCompartments[i], THIS-

>pr_pds);

fMaxPAmbTol = (PAmbTol > fMaxPAmbTol) ? PAmbTol : fMaxPAmbTol;

}

return fMaxPAmbTol;

}

TABLE 6B
float body PAmbTolEval (CBody* pbody, CEnvironment* penv, CBreathingEquipment* pequip, float
fTime)
{

CBody simbody = *pbody; // simbody will share the same Buhlmann tissue threshold model

instance

| CEnvironment simenv = *penv; | |
|---|---|
| ADJUSTSURFACE(simenv); // surface adjustments for safety | |
| CDiveStatus ds = *simbody.pr_pds; | |
| simbody.pr_pds = &ds; | |
| envChangeTime(&simenv, +(signed long int)(fTime*60)); | // time delta in seconds |
| dsExtendTime(simbody.pr_pds, (unsigned short int)fTime); | // time in minutes |

bodyUpdateTissues(&simbody, &simenv, *pgasmix);
return bodyGetToleratedPressure(&simbody) − envGetSurfacePressure(&simenv);

}

where
EQUIP_GET_INSPIRED_GAS(equip) provides the gas mix currently being breathed from the given equipment;
EQUIP_CALCULATE_INSPIRED_GAS(equip, Pressure, DiveStatus) provides the gas mix that will be breathed at the given ambient pressure and dive status for the given equipment. This includes the Dive Control Parameter (DCP) and set-point calculation discussed below; and
ADJUSTSURFACE(simenv) limits the surface pressure in the environment instance to lbar (maximum) and scales it down to 99.5% for safety.

### CNS Model

The CNS Model updates the CNS % and CNS % increase rate of the body instance by the operation in Table 7.

TABLE 7
CBody::UpdateCNS(CEnvironment *penv, CGasMix gas)

float fPPO2 = envGetAmbientPressure(penv) * *gmGetO2Frac(&gas);
float fExposureTimeMins = DIFF_TIME_SECONDS(*envGetRunTimeRef(penv), THIS−>pr_runtimeCNS) / 60;
int i;
for (i = 0; fPPO2 > fCNSLookupTable[i][0] && i < NUMCNS; i++);
if (i==0) {

THIS−>pr_fCNSPerc /= exp(CNS_HALF_TIME * fExposureTimeMins);
THIS−>pr_fCNSIncreaseRate = 0.0;

} else if (i < NUMCNS) {

THIS−>pr_fCNSIncreaseRate = fCNSLookupTable[i][1];
THIS−>pr_fCNSPerc += (fCNSLookupTable[i][1] * fExposureTimeMins);

} else {

// Failure case where PPO2 > fCNSLookupTable[NUMCNS−1][0]
THIS−>pr_fCNSIncreaseRate = CNS_RATE_VLARGE;
THIS−>pr_fCNSPerc = CNS_HUGE;

}
if (THIS−>pr_fCNSPerc < 0.00) THIS−>pr_fCNSPerc = 0.0;

where;
fCNSLookupTable[i][j] is a look-up table of CNS % increase rate (per minute, index j) against PPO₂ (in bar, index i); and
CNS_HALF_TIME is 0.6935/90.0 mins

A Resource Management System (RMS) in the Handset is designed to provide dives that are optimized to maximize certain parameters. The Resource Management System (RMS) prime function is to control the PPO₂. In addition it monitors a range of dive parameters providing a diver with advanced decisions based on available resources in order to modify the PPO₂. The RMS monitors resources including dive depth, No Decompression Limit (NDL), PPO₂, PCO₂, Battery life, High Pressure (HP) gas, Central Nervous System (CNS, oxygen toxicity), and CO2 cartridge filter life.

The design of the RMS assumes the following parameters for the resource management system. For Battery Life, it is assumed usage at a rate of 0.1% per minute from the lowest of 4 Lithium-Ion batteries. Filter usage is assumed to be 1% per minute with a threshold of 10%. HP assumes 1 bar per minute usage with a threshold of 30 bar (the ‘reserve’), and the CNS assumes the current % per minute (increase) rate based on the CNS calculation above. The threshold is 100%.

The RMS employs a breathing detection routine as part of the pre-dive sequence ‘pre-breathe.’ This prevents the diver from doing an incomplete filter test. Once started, the sequence continually checks to see if breathing has started. If breathing is not detected within 120 seconds, the test fails and the diver can either abort the Pre-Breathe test or restart the 120 second timer to detect breathing. Once breathing is detected, a 60 second timer is started. If breathing continues for the full 60 seconds, the Pre-Breathe test is passed. Whereas, if breathing during the 60 seconds is stopped (not detected—the O₂ metabolism rate is less than 0.0005 bar/min for 5 consecutive seconds) the pre-breathe test failed. At this point, the diver may either decide to abort the Pre-Breathe test or restart the test to continue the pre-dive sequence.

In addition to the pre-dive breathing detection, the electro-mechanical rebreather has an auto wake function at depth. That is, the electro-mechanical rebreather is ‘awaken’ by the handset if the prior state of the rebreather was ‘sleeping’ and the pressure sensor detects an absolute pressure between 1.15 bar and 28 bar.

Further, HP usage is monitored such that a reference pressure and time are maintained. For each drop in HP (via the wireless sender/receiver), usage is determined in bar/min. For every increase in HP, usage is reset to 0 and the reference pressure and time are reset.

Furthermore, depth is recorded every second. A buffer of 12 samples (12 seconds) is maintained and used to form a depth rate over the last 12 seconds and is converted to m/min.

An additional safety feature is a Dive Hours Service Lock. The electro-mechanical rebreather system has 200 hours dealer maintenance free life. A timer counts ‘dive minutes remaining’ from 200 hours to zero to force a dealer service. At zero, a system alarm is raised which prevents entering the pre-dive sequence from the system checks. The alarm is displayed as a red heads up display (HUD) with “SERVICE! DO NOT DIVE! !” action. Although the dive screen cannot be entered, the status wheel on the splash screen will show the action.

LSS Module

The Life Support System (LSS) module allows for the recording and control of the following resources and or functions.

The Filter (CO₂ cartridge) percent usage is determined from a mass counter. The LSS module records every 100 ms the LP solenoid valve is open as a mass counter, also referred to as a click counter. The mass counter is only reset by user action towards the end of a pre-dive sequence in response to an inquiry whether a new CO₂ filter was fitted. The mass counter is used to measure the mass of metabolized O₂, and therefore mass of CO₂ removed from the loop and therefore filter percentage is defined as

Filter %=100−(Mass Counter/100),

where 10,000 clicks are assumed per filter.

The LSS module controls the Nitrox Injection System by controlling an HP solenoid valve to vary the gas introduced into the breathing loop. This is determined based on the PPO₂ level in the loop and availability of additional information from the handset. In some circumstances, fail-safe injection modes are entered that don't use handset data or PPO₂ level. Such fail-safe injection modes arise when the system LSS cannot determine PPO₂ level, such as when handset communication is lost by the LSS module or when all sensor module communication is lost or a closed-circuit bail-out condition has been determined. In such situations, the LSS module will inject for 1 second every 3 seconds, allowing a diver to safely ascend.

PPO₂

The Sensor module which comprises 3 O₂ sensors and 1 CO₂ sensor, reports three separate PPO₂ cell mV levels to the LSS module. These levels are used to determine a single PPO₂ value (in bar) plus alarm states. Depending on the reading of a particular sensor, the sensor may be removed from the averaging or a warning issued to the diver. For example, if a single sensor is below 0.15 bar or above 3.00 bar, then that particular sensor is removed from the averaging. Similarly, a sensor is removed if the sensor is less than 7 mV. If one sensor is +/−0.2 bar away from the two remaining sensors then it will be removed from the averaging. If the difference between the highest and the lowest is greater than 0.5bar then the system will inject gas for 1 second out of every 3 as a fail-safe. If any sensor is greater than 1.6 bar when diving then an ASCEND NOW alarm will be displayed. If any sensor is less than 0.17 or greater than 2.0 when diving then a BAILOUT alarm will display. If two or more sensors are removed from the averaging then the system will inject gas for 1 second out of every 3 as a fail-safe. This will cause an ASCEND NOW alarm.

The following description refers to a single quantity for PPO₂ as determined by the sensor rules.

The maximum theoretical partial pressure of oxygen, or PPO₂, available to the diver in the breathing loop at any depth is calculated as the product of the concentration of oxygen, or FO₂, in the oxygen-enriched gas mixture supply and the ambient pressure, or Pambient. Because Pambient varies with depth, the partial pressure of oxygen for the gas supply also varies with depth and reaches a maximum value at the maximum depth of a dive. To maintain the maximum partial pressure of oxygen, or PPO₂(max), at safe levels during the course of a dive, in certain embodiments, the diver may select a gas supply for which the maximum partial pressure of oxygen will not exceed a selected threshold. For example, if the gas supply is an oxygen-enriched gas mixture, the desired maximum partial pressure of oxygen for the duration of the dive is 1.6 bar and the planned maximum dive depth is 40 meters, at which Pambient is 5 bar, then the appropriate concentration of oxygen in the oxygen-enriched gas supply will be equal to or less than PPO₂(max)/Pambient=1.6 bar/5 bar=32%. Conversely, a known viable minimum partial pressure of oxygen is 0.21 bar, which corresponds to a concentration of oxygen of 21% at 1 bar. The maximum potential dive depth using an oxygen-enriched gas supply and maintaining at least a known viable minimum partial pressure of oxygen is then determined according to PPO₂(max)/FO₂=1.6/21%=7.6 bar, which corresponds to approximately 66 meters.

Selecting the concentration of oxygen in this manner allows the oxygen-enriched gas supply to be used safely in an open-circuit bailout configuration of the rebreather system, in which a diver would inhale the oxygen-enriched gas supply without the use of automatic control to maintain the partial pressure of oxygen in the breathing loop below a safe level. In the event that the solenoid valve failed during a dive and remained open to continue providing breathing gas to the diver, selecting the concentration of oxygen according to the maximum planned dive depth would also help insure that the constant addition of the oxygen-enriched gas supply to the breathing loop through the open solenoid valve would not produce excessive partial pressure of oxygen in the breathing loop.

The method of the present invention allows the diver to balance gas usage against decompression requirements by maintaining an adjustable setpoint for partial pressure of oxygen that varies with ambient pressure as dive depth varies. In some embodiments, the diver selects a value for a control parameter, a Dive Control Parameter (“DCP”) expressed as a fraction or percentage of the range between the minimum and maximum safe partial pressures of oxygen. In some embodiments, the value of the DCP varies between 10% and 100%.

DCP Modes

There are two DCP modes. As described below, the DCP control can be set to automatic mode and will follow the rules described below or can be set to manual mode and can be adjusted by the diver between 0 and 100%.

In Manual mode, the DCP can be set by the diver at any time on the dive screen of the Handset.

While the DCP can be controlled by the diver manually it can also be set into AUTO mode. The purpose of the Auto DCP mode is to adjust the DCP to achieve an initial NST of 60 minutes after an initial dive time of 10 minutes. For deeper depths, the DCP will run high, for shallower depths, the DCP will run low. This has the advantage of making the NST focused at around 60 minutes for as wide a range of depths as possible. Where the depth is shallow, this will also result in saving of resources. Where the depth is deeper, this will result in increased resource use, but giving an improved NST. The DCP will be increased in 10% increments to a maximum of 95% before 10 minutes into the dive and then be optionally reduced in 5% decrements if the current NST allows.

In Auto Mode, when the NDL (no decompression limit) remaining reaches a preset number, the DCP automatically adjusts (as time at depth continues) to make the NDL remain at the preset figure, continually reducing the DCP until a minimal safe (pre-determined) PO₂ is reached. At this point, while the NDL could still increase due to the depth of dive and the tissue loading, the DCP will not reduce further.

The electro-mechanical rebreather system can also automatically adjust the setpoint to maintain an NDL maximum of 60 minutes when it can. This means that at the start of the dive (the deeper part) as the NDL will naturally reduce as the time increases at depth, the DCP will inject as much gas as possible (raise the setpoint) to keep the NDL to a maximum. As the diver ascends and the NDL naturally increases it will limit at 60 minutes and then start to reduce the setpoint/DCP to keep it at 60 minutes maximum for the remainder of the ascent. The only exception being that if there is decompression to do, then the DCP/setpoint will stay high until the decompression is complete and then it will reduce again based on the first rule for the remainder of the ascent.

In the Auto mode, the DCP is calculated as follows.

The system defaults back to auto-mode and 50% DCP on “system wake”. The diver is provided the option to go into manual mode at any time from the dive screen. If in auto-mode, on detection of >10 m depth (for the first time each dive), the DCP is set to 95%. Diving in auto-mode (after first detection of >10 m depth), every 30 s or (if longer) the next time the decompression thread evaluates (min 1 hz):

TABLE 8
if (NST < 60mins and DCP <= 85%) // NST is the current no-stop-time in
minutes

DCP = DCP + 10%

else if (DT >= 10mins and NST > 60mins and DCP >= 15%) // DT is the
current dive-time

DCP = DCP − 5%

end if

The setpoint control system uses a variable Nitrox injection at 3 second intervals to maintain the optimum PPO₂ level in the breathing loop.

Inject Time=max(min(20*(SP−PPO₂), 1.2),0) seconds

where max(a,b) selects the larger of a and b.

The above algorithm is the nominal case where all required communications and sensor readings are available.

A diver may select the maximum value of the Dive Control Parameter for the purpose of minimizing decompression time during the dive-ending ascent. Choosing the maximum value of the Dive Control Parameter would induce the control system to add the gas supply to the rebreather loop in order to maintain a higher target partial pressure of oxygen (closer to the maximum theoretical partial pressure of oxygen value). The increased volume of gas addition may cause increased venting gas from the rebreather loop and higher usage rates of the gas supply. A resulting penalty of choosing the maximum Dive Control Parameter value may therefore be less efficient use of the gas supply.

The above effect is amplified as depth, and ambient pressure, increase. The maximum theoretical partial pressure of oxygen is directly proportional to ambient pressure, and the target partial pressure of oxygen increases with the maximum theoretical partial pressure of oxygen at a constant control parameter value. Accordingly, as depth increases, the rebreather control loop increases the addition of the gas supply to the rebreather loop to maintain the target partial pressure of oxygen, which may produce excessive total gas pressure in the rebreather breathing loop. Excess gas is vented, or purged, automatically from the rebreather breathing loop to maintain total gas pressure in the rebreather loop below a preselected threshold.

Conversely, a diver may select the minimum value of the Dive Control Parameter for the purpose of minimizing the venting of gas from the rebreather loop and minimizing the consumption of the gas supply. However, by specifying the minimum Dive Control Parameter value, the diver biases the operation of the rebreather system to maintain a lower target partial pressure of oxygen, which increases the required decompression time.

At various times during a dive, the diver may wish to either minimize gas venting from the rebreather breathing loop or minimize decompression time. The inventive method and control system enable the diver to increase or decrease the Dive Control Parameter and the partial pressure of oxygen while a dive is in progress, while staying within safety limits, in order to accommodate changing objectives.

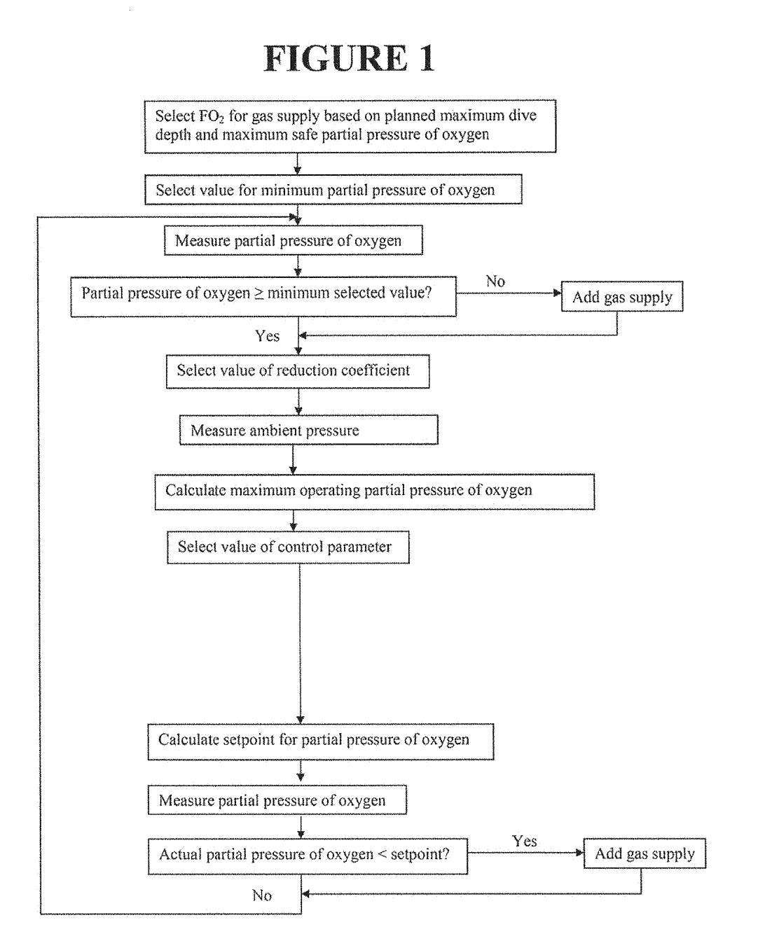

FIG. 1 illustrates a method of automatically controlling partial pressure of oxygen in accordance with the present invention for a rebreather configuration using an oxygen-enriched gas supply. A setpoint for the partial pressure of oxygen may be calculated according to the following equations:

S=P*(R*PPO₂₍ₘₐₓ₎−PPO₂₍ₘᵢₙ₎₎+PPO₂₍ₘᵢₙ₎PPO₂₍ₘₐₓ₎=FO₂*Pambient

where S is the setpoint for the partial pressure of oxygen, P is the Dive Control Parameter, R is the reduction coefficient. PPO₂₍ₘₐₓ₎ is the maximum value of partial pressure of oxygen at a specified depth, PPO₂₍ₘᵢₙ₎ is the selected minimum partial pressure of oxygen, FO₂ is the concentration of oxygen in the gas supply and P_{ambient} is ambient pressure.

The oxygen partial pressure setpoint in a prophetic example with FO₂=40%, P_{ambient}=3 bar at 20 meters depth. PPO₂₍ₘᵢₙ₎=0.3 bar, R=0.9 and P=50% will be

PPO₂₍ₘₐₓ₎=3 bar*40%=1.2 bar

S=0.5*(0.9*1.2 bar−0.3 bar)+0.3 bar=0.69 bar

In a rebreather configuration using a diluent gas and a gas supply containing pure oxygen, the concentration of oxygen in the gas supply would be 100%. Accordingly, it would be possible for the maximum theoretical partial pressure of oxygen to exceed a viable safe limit of for example, 1.6 bar. Therefore, the method illustrated in FIG. 1 incorporates an additional step by which, if S, calculated in accordance with the equations listed above, exceeded a viable safe limit for partial pressure of oxygen during a dive, then the value of S would be set at the viable safe limit.

In accordance with this method, the setpoint is adjusted to a partial pressure of oxygen that can be maintained within the limits established by safety considerations and depth, and the Dive Control Parameter may be varied to suit the dive plan and yield a setpoint that balances decompression time and gas usage. If the partial pressure of oxygen in the rebreather breathing loop is below the setpoint, a portion of the gas supply is added to the rebreather breathing loop to increase the partial pressure of oxygen to the setpoint. In some embodiments, the gas supply will be added to the rebreather breathing loop in pulses of 1 liter per second. If the partial pressure of oxygen in the rebreather loop is above the setpoint, no action will be taken by the control system and excess oxygen will be metabolized by the diver until the partial pressure of oxygen decreases to the setpoint. Because of the diver's metabolism, the diver either consumes more oxygen or less depending on the workload. Metabolism may be represented by the amount of oxygen converted to CO₂. The more oxygen is converted, the less amount of gas (oxygen) in the breathing loop and therefore the partial pressure of oxygen drops. As the partial pressure drops below the target PO₂ level, the system will inject more oxygen into the breathing loop to adjust the PO₂ level.

One of ordinary skill in the art will understand that a control system of the present invention will maintain partial pressure of oxygen in the rebreather loop within a small range relative to the setpoint. In certain embodiments, partial pressure of oxygen in the rebreather loop will be controlled to the value of the setpoint ±0.05 bar.

In accordance with the method shown in FIG. 1, measured data is updated by the processor of the present invention at a rate specified by the control loop. The method in accordance with the present invention may be executed repeatedly at a rate sufficient to ensure that the partial pressure of oxygen in the breathing loop will remain in a viable range to ensure diver safety. In some embodiments, data measurements and calculations will be performed once per second, which is reflective of well-known response times for changes in partial pressure of oxygen in the rebreather loop. One with skill in the art will understand that the parameters of measuring rate and control loop execution rate can be adjusted within limits suitable to maintain partial pressure of oxygen in the breathing loop within safe limits.

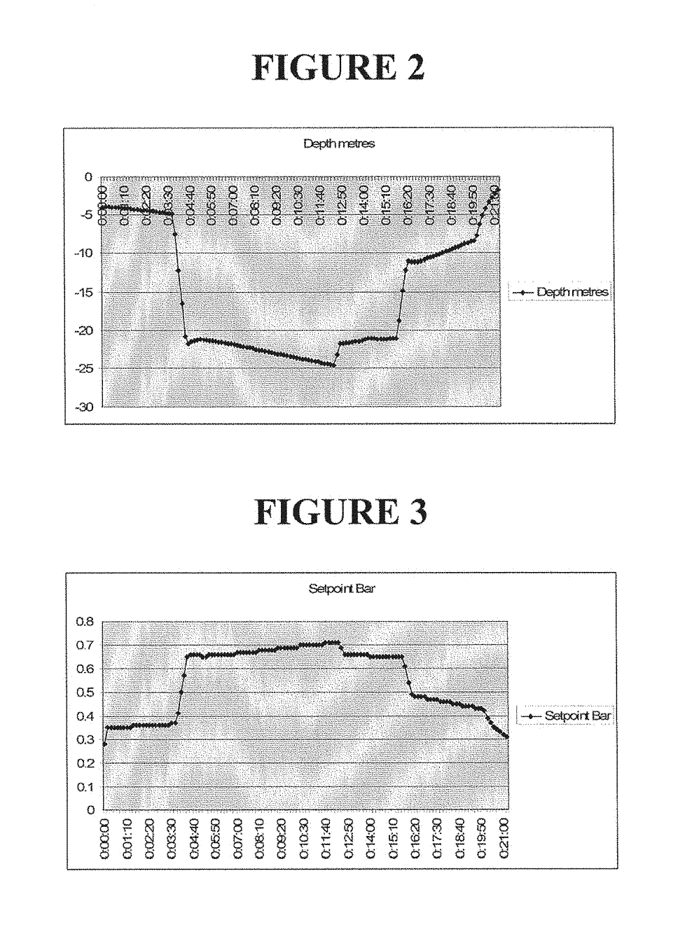

The dependence of oxygen partial pressure setpoint and measured partial pressure of oxygen on dive depth and the control error difference between setpoint and measured partial pressure is shown in FIGS. 2, 3, 4 and 5. These graphical representations display data collected during a test dive conducted using a mechanical breathing apparatus made by Ansti Test Systems Ltd., with the following test conditions: FO₂=40%, PPO₂(min)=0.3 bar, R=0.9 and P=50%.

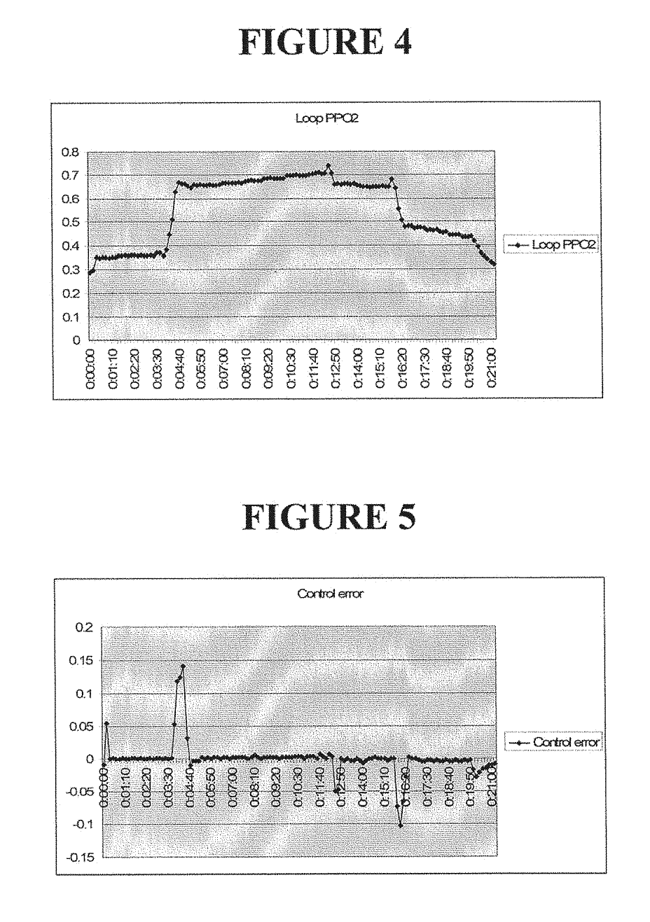

In this working example, the setpoint S was continuously automatically adjusted corresponding to the changes in depth, as shown in FIGS. 2 and 3, and the partial pressure of oxygen in the rebreather loop as shown in FIG. 4 was controlled to the value calculated for S. FIG. 5 depicts the lag exhibited in the control system when depth and ambient pressure changed significantly in a short time period. Increasing depth caused the oxygen partial pressure setpoint to increase, and the oxygen partial pressure measured in the rebreather loop temporarily rose above the setpoint as addition of the gas supply continued after depth ceased to change. Decreasing depth correspondingly caused the oxygen partial pressure measured in the rebreather loop to temporarily drop below the setpoint.

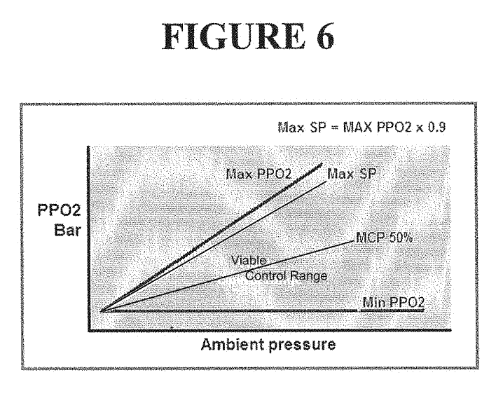

As shown in FIG. 6, use of the present invention enables the diver to optimize gas usage by adjusting the control system within limits that are safe but are wider than would be available with a rebreather system that controls to a constant concentration of oxygen or partial pressure of oxygen in the rebreather loop. The wider range is determined by the ambient pressure, as well as the metabolic rate of the diver and the concentration of oxygen in the gas supply. In particular, a control system in accordance with the present invention will be more efficient compared to a rebreather system that controls to a constant concentration of oxygen because an acceptable partial pressure of oxygen can be maintained in the rebreather loop at shallower depths, where the ambient pressure is lower, using a lower rate of addition of gas supply.

A control system in accordance with the present invention will perform most efficiently if the partial pressure of oxygen is allowed to vary in conjunction with ambient pressure and diver metabolic rate. The overall allowable range of partial pressure of oxygen would need to ensure that partial pressure of oxygen does not fall below safe levels required for metabolism, and does not rise above levels associated with central nervous system toxicity. Within this wide operating range, the diver narrows the range to improve predictable operation of the rebreather system by choosing a value of the control parameter P that sets the bias of operation from conservative use of supply gas and low venting to high gas supply usage with higher partial pressure of oxygen and reduced potential for decompression requirements.

Another aspect of the present invention is the management of dive resources, dive resource usage rates, dive resource net effects, dive variables, dive time limitations and pre-selected dive plans. The viability of a pre-selected dive plan is determined by the types of factors shown in Table 9.

TABLE 9

| Type | Description |
|---|---|
| Dive resource | A capacity of the diving apparatus that can be |
| | measured or is known, and may be replenished |
| Dive resource | Change in dive resource over time |
| usage rate | |
| Dive resource | Expected duration of a remaining dive resource |
| net effect | based on usage rage |
| Dive variable | An environmental factor affecting decompression |
| | time, no decompression limit or central |
| | nervous system oxygen toxicity percentage |
| Dive time | A dive requirement determined by decompression |
| limitation | time, no decompression limit or central |
| | nervous system oxygen toxicity percentage |
| Dive plan | May comprise a target dive time and/or |
| | preselected dive-end values for one or more |
| | dive resources |

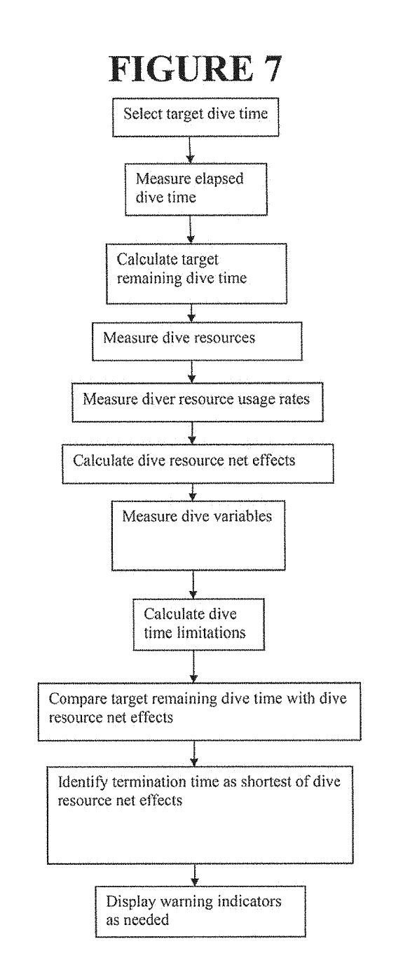

As shown in FIG. 7, dive resources, dive resource usage rates, dive resource net effects, dive variables and dive time limitations may be evaluated continuously to advise the diver regarding the status of the dive and to warn the diver if a pre-selected dive plan becomes non-viable. A processor configured in accordance with certain embodiments can calculate or receive data corresponding to, for example, pressure of a gas supply, scrubber capacity, battery capacity, decrease in pressure of the gas supply over time, number of injections from the gas supply into the rebreather breathing loop over time, electrical current, gas supply duration, scrubber canister duration, battery duration; depth, elapsed dive time, partial pressure of oxygen in the rebreather breathing loop, no decompression limit decompression time, central nervous system oxygen toxicity percentage, concentration of carbon dioxide in the rebreather breathing loop and temperature of the scrubber canister where a sensor assembly comprising of one or more sensors is adapted to measure temperature of the scrubber canister.

One of skill in the art will understand that dive resources, dive resource usage rates, dive resource net effects, dive variables and dive time limitations may be calculated and/or measured through suitable means known in the art. For example, no decompression limit and decompression time can be determined through using dive tables compiled by the United States Navy or tables derived from the Buhlmann decompression algorithm of other suitable decompression algorithms.

In a method in accordance with certain embodiments, a dive plan and resource changes are input by a user, and the duration remaining for each dive resource is determined from the dive resource measurement data and the calculated or measured usage rates. The dive resource having the shortest duration is identified as the controlling resource at that stage in the dive, and the duration to the completion of the dive plan or a segment of the dive plan is measured against the dive resource time remaining. If the duration of the identified dive resource is shorter than the duration to the completion of the dive plan or segment, then the duration of the identified dive resource and the dive resource type are displayed.

The viability of the dive plan or segment is determined based in part upon decompression requirements, no-decompression limit and dive segment target time. The dive plan or segment is
1. Viable, with no use of reserve resources;
2. Viable, with partial use of reserve resources; or
3. Not viable, exceeding available dive resources and reserve resources.

In certain embodiments, the processor and rebreather system display a bar graph of the dive resource having the shortest duration and a graphical indication of the dive viability. In certain embodiments, the processor and rebreather system display warning indicators corresponding to each category of viability. In certain embodiments, the warning indicators are color coded, such as green for category 1, yellow for category 2 and red for category 3.

In certain embodiments, the processor and rebreather system display a time countdown to dive resource completion for the dive resource having the shortest duration. In certain embodiments, the processor and rebreather system display an overrun time to indicate the magnitude by which the dive resource usage exceeds the duration of the dive resource so that the diver can take appropriate action.

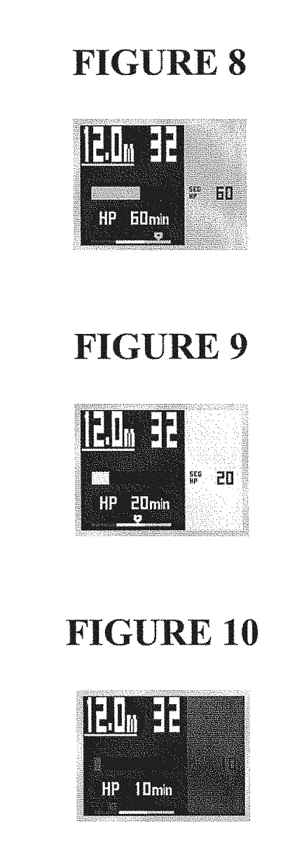

FIGS. 8, 9 and 10 provide exemplary graphical representations of the warning indications and duration data that may be displayed by a processor and rebreather system in accordance with certain embodiments. In the scenario depicted in FIG. 8, the gas supply is sufficient to complete the dive. Gas supply duration is less than other dive resources and the no-decompression limit. In this embodiment, the rebreather system displays green color-coding and the diver may continue the dive.

In the scenario depicted in FIG. 9, the gas supply is not sufficient to complete the planned dive without consuming reserve supply gas. In this embodiment, the rebreather system displays yellow color-coding, to warn the diver that the planned dive is not viable without using reserve dive resources.

In the scenario depicted in FIG. 10, the planned dive requires dive resources that exceed the sum of the primary gas supply and the reserve gas supply. In this embodiment, the rebreather system displays red color-coding to warn the diver that the planned dive is not viable and the diver should ascend.

Table 10 shows a prophetic example of dive resources, dive resource usage rates, dive resource net effects, dive variables and dive time limitations in which no decompression limit is less than the available dive resource net effects. Accordingly, the remaining available dive time is equal to the no decompression limit. All duration calculations are measured in minutes to the end of the dive resources and the beginning of reserve resources.

TABLE 10

| Dive Resource | Quantity | | Dive Resource Net |
|---|---|---|---|
| or Dive Time | or Duration | Rate of | Effect or Dive |
| Limitation | Remaining | Consumption | Time Limitation |
| Gas supply | 78 bar | 1 bar per min | 78 min |
| Scrubber duration | 436 gas | 5 injections | 86 min |
| | injections | per min | |
| No decompres- | | | 67 min |
| sion limit | | | |
| Battery capacity | | | 340 min |
| Dive time | | | 67 min |
| remaining | | | |

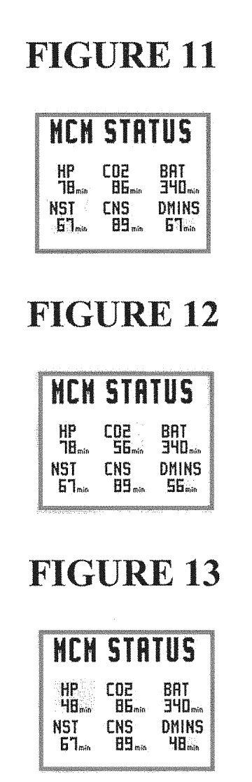

FIG. 11 shows a prophetic example of a graphical representation of information in Table 3 as displayed to the diver.

Table 11 shows a prophetic example of dive resources, dive resource usage rates, dive resource net effects, dive variables and dive time limitations in which scrubber duration is the dive resource net effect or dive time limitation of shortest duration. Accordingly, the remaining available dive time is equal to the scrubber duration. All duration calculations are measured in minutes to the end of the dive resources and the beginning of reserve resources.

TABLE 11

| Dive Resource | Quantity | | Dive Resource Net |
|---|---|---|---|
| or Dive Time | or Duration | Rate of | Effect or Dive |
| Limitation | Remaining | Consumption | Time Limitation |
| Gas supply | 78 bar | 1 bar per min | 78 min |
| Scrubber duration | 280 | 5 injections | 56 min |
| | injections | per min | |
| No decompres- | | | 67 min |
| sion limit | | | |
| Battery capacity | | | 340 min |
| Dive time | | | 56 min |
| remaining | | | |

FIG. 12 shows a prophetic example of a graphical representation of information in Table 11 as displayed to the diver.

Scrubber duration is not affected by the selected value of the control parameter P and instead is inversely proportional to diver metabolic rate. Increasing depth generally induces higher diver metabolic rate. Although diver metabolic rate is not a variable controlled by the rebreather system, carbon dioxide concentration and rate of addition can be measured to inform the diver of the quality of the breathing gas and warn the diver of decreasing scrubber duration.

Table 12 shows a prophetic example of dive resources, dive resource usage rates, dive resource net effects, dive variables and dive time limitations in which gas supply is the resource of shortest duration. Accordingly, the remaining available dive time is equal to the gas supply duration. All duration calculations are measured in minutes to the end of the dive resources and the beginning of reserve resources.

TABLE 12

| Dive Resource | Quantity | | | Dive Resource Net |
|---|---|---|---|---|
| or Dive Time | or Duration | | Rate of | Effect or Dive |
| Limitation | Remaining | | Consumption | Time Limitation |
| Gas supply | 48 | bar | 1 bar per min | 48 min |
| Scrubber duration | 436 | | 5 injections | 86 min |
| | injections | | per min | |
| No decompres- | 67 | min | | 67 min |
| sion limit | | | | |
| Battery capacity | | | | 340 min |
| Dive time | | | | 48 min |
| remaining | | | | |

FIG. 13 shows a prophetic example of a graphical representation of information in Table 14 as displayed to the diver.

Table 13 shows a prophetic example of dive resources, dive resource usage rates, dive resource net effects, dive variables and dive time limitations in which additional dive time is viable in excess of the required decompression time. All duration calculations are measured in minutes to the end of the dive resources and the beginning of reserve resources.

TABLE 13

| Dive Resource | Quantity | | | Dive Resource Net |
|---|---|---|---|---|
| or Dive Time | or Duration | | Rate of | Effect or Dive |
| Limitation | Remaining | | Consumption | Time Limitation |
| Gas supply | 200 | bar | 1 bar per min | 200 min |
| Scrubber duration | 500 | | 5 injections | 100 min |
| | injections | | per min | |
| No decompres- | 60 | min | | 60 min |
| sion limit | | | | |
| Battery capacity | 3000 | mA-hr | 30 mA | 6000 min |
| Additional dive | | | | 40 min |
| time available | | | | |
| at depth | | | | |

Table 14 shows a prophetic example of dive resources, dive resource usage rates, dive resource net effects, dive variables and dive time limitations in which the shortest duration dive resource, specifically, scrubber canister duration, is equal to required decompression time. In this exemplary scenario, continuing the dive would not be viable and the diver would need to begin the ascent, including decompression stops, immediately. All duration calculations are measured in minutes to the end of the dive resources and the beginning of reserve resources.

TABLE 14

| Dive Resource | Quantity | | | Dive Resource Net |
|---|---|---|---|---|
| or Dive Time | or Duration | | Rate of | Effect or Dive |
| Limitation | Remaining | | Consumption | Time Limitation |
| Gas supply | 200 | bar | 1 bar per min | 200 min |
| Scrubber duration | 500 | | 5 injections | 100 min |
| | injections | | per min | |
| No decompres- | 100 | min | | 100 min |
| sion limit | | | | |
| Battery capacity | 3000 | mA-hr | 30 mA | 6000 min |
| Additional dive | | | | 0 min |
| time available | | | | |
| at depth | | | | |

Table 15 shows a prophetic example of dive resources, dive resource usage rates, dive resource net effects, dive variables and dive time limitations in which scrubber duration based on volume of gas supply usage is the resource of shortest duration. Scrubber duration exceeds target dive time, and additional dive time at depth is available. All duration calculations are measured in minutes to the end of the dive resources and the beginning of reserve resources.

TABLE 15

| Dive Resource | Quantity | | | Dive Resource Net |
|---|---|---|---|---|
| or Time Dive | or Duration | | Rate of | Effect or Dive |
| Limitation | Remaining | | Consumption | Time Limitation |
| Gas supply | 200 | bar | 1 bar per min | 200 min |
| Gas supply | 200 | bar | 1.6 liter | 130 min |
| (metabolic rate) | | | O₂ per min | |
| Scrubber duration | 500 | | 5 injections | 100 min |
| | injections | | per min | |
| Scrubber duration | | | | 120 min |
| (thermal profile) | | | | |
| Scrubber duration | <0.4 | mB | | |
| (carbon dioxide | | | | |
| concentration) | | | | |
| Diver metabolic | 1.2 liter | | | |
| rate | O₂ per min | | | |
| Battery capacity | 3000 | mA-hr | 30 mA | 6000 min |
| No decompres- | 90 | min | | |
| sion limit | | | | |
| Decompression | none | | | |
| time | | | | |
| CNS toxicity | 10% | | 0.5% per min | 140 min |
| percentage | | | | |
| Target dive time | | | | 80 min |
| Additional dive | | | | 20 min |
| time available | | | | |
| at depth | | | | |

Table 16 shows a prophetic example of dive resources, dive resource usage rates, dive resource net effects, dive variables and dive time limitations in which scrubber duration based on volume of gas supply usage is the resource of shortest duration. Target dive time exceeds scrubber duration. Accordingly, continuing the dive would not be viable and the diver would need to begin the ascent, including decompression stops, immediately. All duration calculations are measured in minutes to the end of the dive resources and the beginning of reserve resources.

TABLE 16

| Dive Resource | Quantity | | | Dive Resource Net |
|---|---|---|---|---|
| or Time Dive | or Duration | | Rate of | Effect or Dive |
| Limitation | Remaining | | Consumption | Time Limitation |
| Gas supply | 200 | bar | 1 bar per min | 200 min |
| Gas supply | 200 | bar | 1.6 liter | 130 min |
| (metabolic rate) | | | O₂ per min | |
| Scrubber duration | 500 | | 5 injections | 100 min |
| | injections | | per min | |
| Scrubber duration | | | | 120 min |
| (thermal profile) | | | | |
| Scrubber duration | <0.4 | mB | | |
| (carbon dioxide | | | | |
| concentration) | | | | |
| Diver metabolic | 1.2 liter | | | |
| rate | O₂ per min | | | |
| Battery capacity | 3000 | mA-hr | 30 mA | 6000 min |
| No decompres- | 90 | min | | |
| sion limit | | | | |
| Decompression | none | | | |
| time | | | | |
| CNS toxicity | 10% | | 0.5% per min | 140 min |
| percentage | | | | |
| Target dive time | | | | 120 min |
| Additional dive | | | | −20 min |
| time available | | | | |
| at depth | | | | |

Some illustrative implementations of an inventive method for managing dive resources have been illustrated in the foregoing prophetic examples with regard to a rebreather system in accordance with some embodiments of the invention. However, various embodiments of the inventive method for managing dive resources could alternatively be applied to any rebreather or diving system where resources are limited and must be used in combination with the diver's decompression or physiological requirements. Additionally, it will be understood that the displays shown in the drawings (FIGS. 8-13) are merely illustrative; the present invention is not limited to embodiments employing the format, content, graphics, etc., as depicted in these illustrative embodiments. For example, various alternative embodiments may be implemented using a different human-computer interface (HCI), and may present additional or different (e.g., not necessarily all information shown in FIGS. 8-13, etc.) information than presented in the illustrative displays of FIGS. 8-13.

In view of the foregoing, those skilled in the art will understand that some embodiments of the present invention provide for variable electronic control of PO₂, wherein the PO₂ may be varied according to, for example, one or more of the following parameters: (a) depth; (b) no stop time remaining or decompression obligation building (e.g., PO₂ can be increased or reduced); (c) resource limitation (e.g., HP, CO₂, canister duration, oxygen toxicity, water temperature, battery life, remaining bailout gas). Additionally, in accordance with some embodiments, a dive monitoring system not only monitors and displays all the parameters of the dive (e.g.,. depth, time, decompression, oxygen toxicity, gas available, canister duration, CO₂ level, temperature, battery life), but also displays those parameters in a clear manner as a controlling resource time. In some embodiments, this information may additionally be used by the system to modify the PO₂ and/or remaining dive time by generating warnings.

In accordance with embodiment of the present invention, one with ordinary skill in the art will understand that data measurements can be taken and calculations can be performed with regard to dive resources, dive resource usage rates, dive resource net effects, dive variables and dive time limitations at a frequency suitable to insure that the inventive method and apparatus can monitor and indicate rapid changes in any of these quantities.

Additionally, in view of the foregoing disclosure, those skilled in the art will understand that the system may be configured to adjust (e.g., reduce) the control PO₂ (setpoint) based on user input of the control parameter or via automatic adjustment of the control parameter (e.g., initiated by the processor according to program control and not directly in response to user input), or both. Those skilled in the art will also understand in view of the foregoing that various such embodiments of the system can be applied to single gas rebreathers as well as to dual gas rebreathers (e.g., separate HP 100% oxygen and HP diluents gas supplies), such as in a gas extender mode to save oxygen.

By way of example for purposes of clarity of exposition, in some embodiments if the system determines that oxygen is running low and may not be enough to complete the planned dive time, then the system can automatically reduce PO₂ by effectively varying (decreasing) the control parameter. More specifically, for example, the resources management system may elect to automatically modify the control parameter based on remaining HP gas and any decompression requirement. As a specific illustrative example, if the decompression time were 30 minutes and the HP O₂ gas supply were estimated by the resources management system to only last 25 minutes the control parameter could be adjusted automatically to reduce the PO₂ and make the gas supply last longer. It will be understood, however, that reducing PO₂ will increase the decompression time; as such, the resources management system may be configured to balance the increased decompression time against the new gas supply time available to ensure a safe exit from the dive. As indicated, such an illustrative scenario applies to dual and single gas implementations of designs according to various embodiments of the present invention.

It may be appreciated, therefore, that adaptively controlling PO₂ (e.g., rather than controlling PO₂ according to a fixed setpoint) in accordance with some embodiments of the present invention provides for a gas extender mode, wherein HP oxygen usage can be extended. It will also be understood in view of the foregoing, that while gas extender control may be applied to dual gas and single gas systems, it may be more advantageously applied to 100% oxygen in a dual gas system, as less gas injection and venting may occur compared to a single gas (e.g., using a 40% O₂ mix). Similarly, gas extender control typically is more advantageous for higher oxygen gas fractions, as generally a higher FO₂ of the gas supply allows the system to more efficiently control PO₂ to a high level, as with each injection there is less inert gas injected and hence less additional volume to vent.

In accordance with the foregoing, those skilled in the art will understand that adaptively controlling PO₂ and managing dive resources according to various embodiments of the present invention comprises source gas injection into the rebreather loop as well as venting of the rebreather loop in the event that the loop pressure (volume) exceeds a given level. According to some aspects, various embodiments of the present invention include a loop control valve that is operable for venting excessive volume while also compensating for the hydrostatic effect associated with the difference between the pressure exerted on the diver's lung centroid and the pressure exerted on the counterlung.

More specifically, as understood by those skilled in the art, the volume of gas contained within the rebreather exerts a pressure on the diver's lungs and is typically measured at the mouthpiece and, in some instances, is designed to be maintained within, for example, +/−25 mb for the various diver orientations. If a diver has a fixed volume within the rebreather, due to various factors including the orientation, size, and shape of the counterlungs and their ability to move freely within the rebreather design, this volume will exert a changing pressure at the mouthpiece of the diver based on the diver's orientation due to the pressure difference (measured difference) between the counterlung and the diver's lung centroid. This orientation dependent pressure difference is known as the hydrostatic effect.

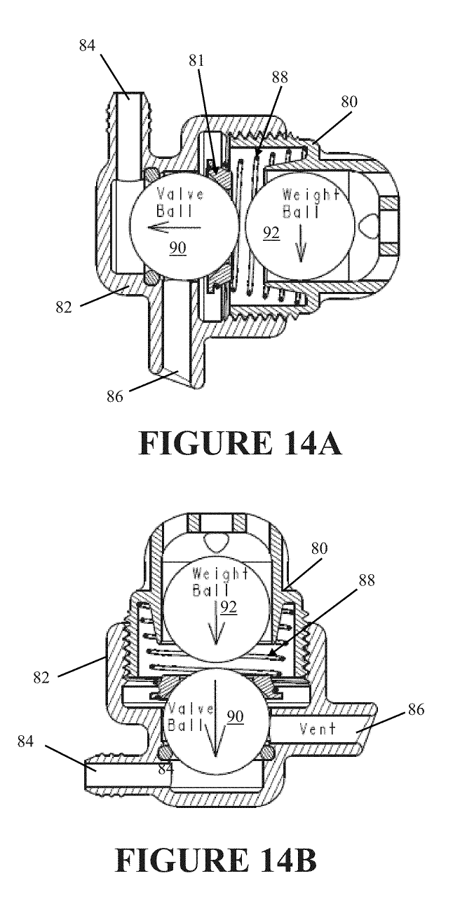

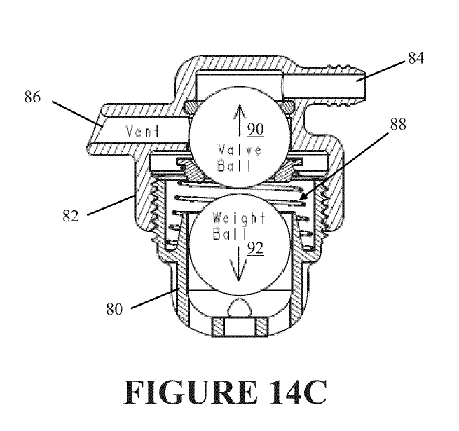

FIGS. 14A-C depict an illustrative loop control valve (LCV) of the present invention that provides for venting loop overpressure while compensating for the hydrostatic effect, in accordance with some embodiments. In accordance with some embodiments, the LCV is configured to regularly vent the excess volume of gas that may arise in accordance with adaptively controlling PO₂ based on a control parameter as discussed above. It will be understood, however, that an LCV as disclosed herein may be implemented in various rebreathers, regardless of the particular PO₂ control method.

Whereas a conventional rebreather typically employs a single overpressure valve, such as a loop exhaust valve comprises a rubber like sealing valve and a sprung plate. The force of the spring acting on the plate and valve control the exhaust pressure of the valve. The plate and spring are only affected by depth pressure such that if they are lower than the diver's lungs the diver has to create a higher pressure to force air out of the valve. If they are higher, then the valve may open easily. Hence while the spring force is constant, the diver's position in the water affects how hard or easy it is to exhaust the loop. This is not the case with respect to the LCV of the present invention, whereby the exhaust pressure is independent of the diver orientation and is near constant.

According to various embodiments employing an LCV such as that depicted in FIGS. 14A-C, the overpressure function is provided by two valves: e.g., the LCV valve of FIGS. 14A-C and a separate overpressure valve (not shown). This latter overpressure valve may comprise, for example, a valve seat and a spring set to approximately 40 mb, such that this overpressure valve acts as a high flow safety valve. The latter overpressure valve may be positioned, along with the LCV, as mechanically close as possible to the diver's lung centroid (e.g., mounted centrally on the diver's back).

As shown in FIGS. 14A-C, LCV comprises a cap portion 80 threadably coupled to main housing 82, which includes a port 84 coupled to the rebreather loop (i.e., at loop pressure) and a vent port 86. The LCV also comprises a valve ball 90, spring 88, and weight ball 92. Balls 90 and 92 may be implemented as rubberized steel balls. The valve seat in the LCV is formed by weighted ball 90 touching the valve walls and the ball is held in place by spring 88. The LCV gets a gas feed from the exhale counterlung via a one-way valve. The LCV and one-way valve combined provide for a regular water removal system from the exhale counterlung; as the LCV vents, water is expelled.

The valve ball 90 and weight ball 92, together with the spring 88, are configured to compensate for the hydrostatic effect: depending on the orientation of the diver, and hence the orientation of the LCV, the balls and spring will result in a different loop pressure threshold being required for the LCV to vent the loop volume. The force of the spring and the weight of the balls are chosen to equate to a certain force/pressure in a given orientation. For a given configuration of ball weights and a given spring, the vent pressure is adjustable via the ramped and castelated adjustment ring 81 (FIG. 14A), which provides for adjusting the spring force by varying the compression on the spring.

It is contemplated that the LCV may utilize one rubberized ball, the valve ball 90. Thus, the LCV may comprise one or more rubberized steel balls.

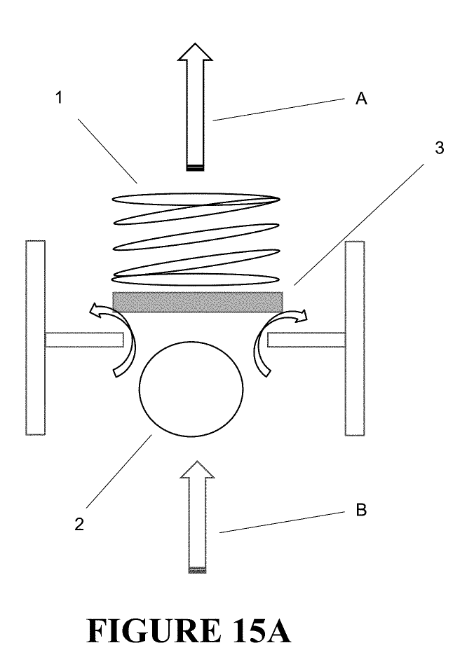

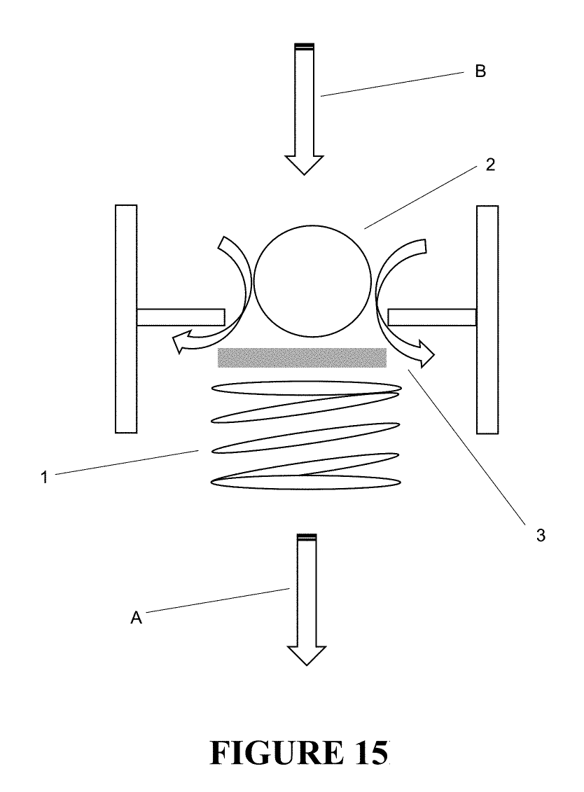

FIGS. 15A and 15B depict the principle operation of the LCV with a single rubberized ball. In a two (2) ball configuration (shown in FIG. 14) one of the two balls is coincident with the spring 22. In general, when a diver swims face down (Mode 1), it is easier breath out and therefore it is desirable to have a greater force acting upon the valve. In contrast, when a diver swims upside down (Mode 2), it is generally harder for the diver to breath out and therefore it is desirable to have a smaller force acting upon the valve, such that it is easier to vent the exhaust gas out. It is desirable to have the release pressure constant in either diver configuration, Mode 1 or Mode 2 or any position in between.

FIG. 15A depicts the operation of the LCV in Mode 1 (diver swimming face down). In this situation, the LCV is on top of the rebreather. The Ball 2 moves away from the Valve 3 due to gravitational forces and the reduced hydrostatic pressure tries to open the valve but the full Spring 1 tries to keep the valve 3 closed. As a result the loop valve vents gas from the rebreather loop B to the water atmosphere A at a pressure difference set by the spring and hydrostatic pressure.

FIG. 15B depicts the operation of the LCV in Mode 2 (diver swimming upside down). In this situation, the LCV is below the rebreather and the hydrostatic pressure tries to close the valve. The weight of the ball (gravitational force), however, exerts a force trying to open the valve. Accordingly, gas from the rebreather loop B vents to the water atmosphere A at the same loop pressure as in Mode 1 due to the additional ball effect.

More specifically, for example, (a) FIG. 14A schematically depicts the LCV oriented according to the diver being oriented upright such that the loop pressure is set by the spring only, as the force of the weight ball 92 is orthogonal to the spring force that acts on the valve ball 90 in a direction perpendicular to the surface normal direction; (b) FIG. 14B schematically depicts the LCV oriented according to the diver being oriented face down such that the loop pressure is set by the spring force plus the weight of both balls 90 and 92; and (c) FIG. 14C schematically depicts the LCV oriented according to the diver being oriented face up such that the loop pressure is set by the difference between the spring force and the weight of the valve ball 90, as the weight ball 92 does not contribute and has no effect as it falls away from the valve.

By way of example, in accordance with an illustrative implementation, the vent pressure for the diver upright orientation in FIG. 14A may be about 15 mb; the vent pressure for the diver face down orientation of FIG. 14B is about 35 mb (i.e., equal to about 15 mb (spring force) plus about 10 mb corresponding to the weight of ball 90 plus about 10 mb corresponding to the weight of ball 92); and the vent pressure for the diver face up orientation of FIG. 14C is about 5 mb (i.e., 5 equal to about 15 mb (spring force) minus about 10 mbar corresponding to the weight of valve ball 90). It will be understood that various alternative implementations of the LCV may provide for different orientation dependent vent pressure values based on implementing a different spring force (e.g., the force of a given spring may be adjusted by adjusting its compression; also, different springs may be used) and/or by varying the weight of the balls.

It is understood that the above illustrative embodiment is configured assuming the counterlungs are disposed on the diver's back. That is, in a face down position, with counterlungs positioned on the divers back, the diver would find it easy to exhale; hence to keep the valve closed, according to the embodiment, one ball acts on the spring for closing the seat plus the additional ball acts on the first ball to provide additional force keeping the valve closed until approximately 35 mb is reached at which point it will vent. When the diver is face up, the reverse is true and the mechanical mass of one ball falls away completely and the remaining ball applies a force to the spring to allow it to release at a lower pressure (5 mb in the specific illustrative example). In the upright position as the balls and spring are horizontal, now virtually the only force acting on the ball to seal the seat is the spring force, keeping the valve closed until about 15 mb is reached. As such, the mechanical configuration of the LCV compensates for the hydrostatic effect.

Further, it is understood that while the illustrative embodiment is configured assuming the counterlungs are disposed on the diver's back, alternative configurations may be implemented to appropriately compensate for the hydrostatic effect for rebreather configurations where the counterlungs are disposed at another position, such as on the diver's chest, over-shoulder, or side-mounted.

In accordance with an alternative implementation of an LCV according to some embodiments, rather than implementing the LCV valve using mechanical components that provide an orientation dependent force on the valve stop, the LCV may be implemented according to an electromechanical control loop. For example, the LCV may be implemented by electronically controlled venting, such as by implementing a solenoid loop vent valve (e.g., a gas solenoid) under control of a processor (e.g., the rebreather computer processor) that is responsive to sensors configured for determining the diver's orientation. In some embodiments, the sensors may be accelerometers (e.g., 4-axis accelerometer) and the processor may (i) determine the diver's orientation based on the accelerometer signals, (ii) determine the vertical displacement of the counterlungs relative to the diver's lung centroid based on the determined diver orientation, (iii) detect the loop pressure (from a pressure sensor), and (iv) control the solenoid valve to vent at a loop pressure threshold that is dependent on the vertical displacement between the counterlungs and the diver's lung centroid. The accelerometers may be integrated into LCV, or may be positioned on one or more other portions of the rebreather and/or the diver.

In more detail, in accordance with the above discussion, while the loop pressure sensor senses pressure in the loop, this pressure is different from the pressure in the divers lungs/mouth because of the water column difference, and this difference changes with angular movement of the diver based on the vertical displacement dependent pressure difference between lung centroid. Accordingly, in some embodiments implementing computer controlled electromechanical venting, an accelerometer can be used such that the computer can define or ascertain the diver's orientation or angular movement and hence can calculate the vertical displacement (e.g., based on trigonometric calculation, such as using the Pythagorean theorem, or an equivalent right-angled triangle calculation using a trigonometric function, or by any appropriate transformation or component determination) and therefore the offset pressure from the loop pressure.

More specifically, in accordance with some embodiments, the computer control system (e.g., comprising a processor) uses the accelerometer to define the diver's orientation in the water and hence calculate a known hydrostatic orientation. To accurately determine the pressure differential between the counterlungs and the diver's lung centroid, the computer control system also has information indicative of the position of the counterlungs with respect to the lung centroid. This information may, for example, be input into the computer as a linear distance, or components relative to a reference frame, or be calculated by the system during a calibration routine that determines the 3D relationship between the accelerometer, lung centroid, and counterlung location. With this calibration information, the computer control system can more accurately compensate for hydrostatic pressure changes experienced by the user between the lung centroid and countelungs as his/her orientation changes. Different configurations for different divers can be stored. Also different configurations for alternative equipment, such as BCD (buoyancy control device) changes, can also be stored and compensated for, and the system may also be calibrated according to individual diver feedback as to how it feels to breath (e.g, easy, OK, difficult) under one or more orientations. As such, in a known position (e.g., face down as an example), the system using the loop pressure sensor, accelerometer and feedback from the diver on how it feels to breath (Easy, OK, Hard) can correct for the centroid position in different divers.

Once the computer can accurately determine the pressure difference between centroid and counterlung(s) in any orientation, then the gas solenoid can be controlled to vent excess pressure from the breathing loop at the appropriate time to compensate for the hydrostatic effect. By way of example, the pressure may normally be set to +15 mb but could be adjustable to suit diver comfort requirements, as noted above. Such compensation may also be implemented in accordance with an appropriate PO₂ control method, such as implementations of the adaptive PO₂ control methods described hereinabove.

Although the present invention has been described in considerable detail with reference to certain embodiments thereof, other embodiments are possible. Therefore, the spirit and scope of the appended claims should not be limited to the description of the embodiments contained therein.

1. A method for automatically controlling partial pressure of oxygen in a rebreather breathing loop comprising:
selecting a minimum value of partial pressure of oxygen;
measuring partial pressure of oxygen in the rebreather breathing loop;
adding a portion of a gas supply to the rebreather breathing loop if partial pressure of oxygen in the rebreather breathing loop is less than the minimum value of partial pressure of oxygen;
selecting a value of a first reduction coefficient;
measuring ambient pressure;
calculating a maximum operating value of partial pressure of oxygen as the product of ambient pressure, concentration of oxygen in the gas supply and the first reduction coefficient;
selecting a value of a first control parameter;
calculating a setpoint for partial pressure of oxygen as the sum of the minimum value of partial pressure of oxygen and the product of the first control parameter and the absolute value of the difference between the maximum operating value and minimum value of partial pressure of oxygen; and
adding gas supply to the rebreather breathing loop if partial pressure of oxygen in the rebreather breathing loop is less than the setpoint for partial pressure of oxygen.

2. The method of claim 1 wherein the value of the first reduction coefficient is from 0.75 to 0.95.

3. The method of claim 1 wherein the value of the first control parameter is from 0% to 100%.

4. The method of claim 1 wherein the gas supply is oxygen further comprising
selecting an absolute maximum value of partial pressure of oxygen; and
limiting the setpoint for partial pressure of oxygen to the absolute maximum value of partial pressure of oxygen.

5. The method of claim 1 wherein the gas supply is an oxygen-enriched gas mixture, further comprising:
selecting a gas supply having a concentration of oxygen for which partial pressure of oxygen in the gas supply will not exceed a selected value at a maximum planned dive depth.

6. An automatic control system for a rebreather breathing loop comprising:
a gas supply;
a gas supply pressure regulator;
a scrubber canister;
a counterlung;
a first sensor adapted to measure ambient pressure;
a second sensor adapted to measure partial pressure of oxygen;
a first valve adapted to add the gas supply to the rebreather breathing loop;
a power source; connecting hoses;
check valves adapted to control the direction of flow of gas in the rebreather breathing loop; and
a processor adapted to
receive data for partial pressure of oxygen in the rebreather breathing loop, ambient pressure, a selected minimum value of partial pressure of oxygen, a selected concentration of oxygen in the gas supply, a selected value of a first reduction coefficient and a selected value of a first control parameter,
calculate a maximum operating value of partial pressure of oxygen as a function of ambient pressure, the selected concentration of oxygen in the gas supply and the selected value of the first reduction coefficient,
calculate a setpoint for partial pressure of oxygen as a function of the selected value of the first control parameter, the selected value of a first control parameter and the selected minimum value of partial pressure of oxygen,
compare data for partial pressure of oxygen in the rebreather breathing loop with the setpoint for partial pressure of oxygen, and
send a signal to add a portion of the gas supply to the rebreather breathing loop if the partial pressure of oxygen in the rebreather breathing loop is less than the setpoint for partial pressure of oxygen.
